(19) United States
(12) Patent Application Publication
(10) Pub. No.: US 2026/0263226 A1
(43) Pub. Date: Sep. 10, 2026

(54) IMPLANTABLE PROSTHESIS FOR REPLACEMENT OF THE HUMAN KNEE JOINT

(71) Applicant: Rainer BAUMGART, Munich (DE)

(72) Inventor: Rainer BAUMGART, Munich (DE)

(21) Appl. No.: 19/164,970

(22) PCT Filed: Apr. 24, 2024
(86) PCT No.: PCT/EP2024/061203

§ 371 Date: Sep. 12, 2025

(30) Foreign Application Priority Data
Apr. 25, 2023 (EP) 23169657.6

(51) Int. Cl. A61F 2/38; A61F 2/30

(52) U.S. Cl. CPC A61F 2/384; A61F 2/3859; A61F 2/389; A61F 2002/30024; A61F 2002/30354; A61F 2002/30365; A61F 2002/30433

(57) ABSTRACT

An implantable prosthesis for replacement of a human knee joint, including a joint device with a joint head and a joint base interacting with the joint head, which are provided for selectively realizing at least one joint movement in the form of a bending and/or extension movement. The joint head has two first sliding surfaces which are arranged at opposite axial ends of the joint head and are spatially separated from each other by a central joint head section.

The joint base has two second sliding surfaces which are formed axially opposite each other on laterally arranged joint elements of the joint base and are in sliding engagement with the respective first sliding surfaces of the joint head. The two first sliding surfaces and the two second sliding surfaces are formed to realize at least one bending and/or extension movement, and wherein the central joint head section has at least one guide channel for passing a tool, a distraction intramedullary nail or a femoral or tibial shaft anchor.

### FIELD OF THE INVENTION

The invention relates to an implantable prosthesis for replacement of the human knee joint and, if necessary, also the adjacent bone sections.

### BACKGROUND

Malignant bone tumors are often located near the knee joint on the femur or on the tibia, so that after removal of the affected bone section, both part of the shaft and the associated femoral or tibial joint portion must be replaced by an implantable prosthesis.

The removed part of the shaft of the affected bone is replaced by a corresponding bone shaft replacement element of the prosthesis. The joint portion of the affected bone is replaced by the joint portion of the prosthesis. In each case, a joint portion must also be im-planted in the corresponding bone in order to achieve articulation and coupling with the joint portion of the affected bone. Coupling of the joint portions is effected by a joint device, such as, for example, a hinge or ball mechanism, which is formed to allow a movement between femur and tibia that is as close as possible to the natural movement of the knee joint.

Since natural knee joints allow not only bending but also slight rotation in the transverse plane, it is considered advantageous if knee joint prostheses also offer this possibility. The prosthesis is anchored in the femur and tibia by means of a shaft anchorage inserted into the bone. If removal is planned at a later date, the shaft anchorage can be formed with a smooth surface so that no bone grows into it. If definite anchorage is planned, the surface can be roughened or coated so that bone tissue can grow into it. Alternatively, the shaft anchorage can also be fixed with bone cement.

Since malignant bone tumors often occur in children of growth age, a leg length difference occurs in the further course after surgical removal of the tumor and implantation of a tumor prosthesis due to the loss of the growth plate on the affected side and further growth of the opposite side. The delayed growth primarily affects the bone affected by the tumor itself, but also the corresponding bone, i.e., if the tumor was located on the femur near the knee joint, for example, not only the femur but also the tibia will lag behind in growth. Even if, in this case, the shaft anchorage of the tibia is smooth and polished so that the bone can principally continue to grow, there will still be a growth delay in the corresponding bone, which contributes to the resulting leg length difference.

For replacement of the human knee joint and adjacent bone sections after the removal of bone tumors in children of growth age, prostheses are known which can be used to compensate for leg length differences. Conventional prostheses are either activated from the outside with a tool or have an integrated motor drive so that the shaft replacement element can be extended telescopically. Such prostheses are described, for example, in document EP 2 468 216 B1. Prostheses in which the remaining bone of the body is extended rather than the shaft replacement element are significantly more advantageous.

Possibilities for extending the bone remaining after tumor removal despite a tumor prosthesis are known from EP 1 371 346 A1 and EP 2 468 216 A1. EP 1 371 346 A1 discloses a prosthesis in which the shaft anchorage is replaced by an extension intramedullary nail in the remaining bone, which slowly pulls the two bone fragments apart after osteotomy so that new bone tissue can form in the enlarging gap (callus distraction method). Several extension steps are usually necessary, so that the extension intramedullary nail must be replaced several times. After completion of the extension, the extension intramedullary nail must be replaced with a permanently ingrowing (coated) shaft anchorage. If located in the proximal femur, the replacement is also performed proximally; if located in the distal femur, the re-placement is also performed distally, wherein the joint of the prosthesis only allows this re-placement if the prosthesis is completely disconnected, which significantly increases the risk of infection. The same problem arises if the tumor is located in the proximal tibia or if the tibia needs to be lengthened.

EP 2 468 216 A1 discloses for the first time a solution for replacing the shaft anchorage on the femur using a minimally invasive surgical technique, wherein the components are replaced from the opposite end of the bone. If the tumor is located on the femur and there is no length difference in the lower leg, the use of a prosthesis of the type described in EP 2 468 216 A1 can avoid repeated, extensive surgical access via the prosthesis, thereby significantly reducing the risk of infection.

If the tibia needs to be lengthened, either after primary tumor involvement of the tibia or if the tibia is also shortened after tumor localization in the femur and therefore the temporary shaft anchorage in the tibia is to be replaced by an extension intramedullary nail and, after growth has ceased, the extension intramedullary nail is to be replaced by a coated shaft anchorage, which grows permanently into the bone, the prosthesis must still be disconnected each time, requiring extensive surgical access with a considerable risk of infection.

The advantage described in EP 2 468 216 A1 therefore ultimately only comes into play if there is no simultaneous shortening of the lower leg, as the solution described in EP 2 468 216 A1 cannot be applied to the lower leg. However, in the frequent case where, as already mentioned, the shortening also affects the lower leg, the technique described in EP 2 468 216 A1 does not allow for lengthening without decoupling the prosthesis using a minimally invasive technique. Thus, the advantageous possibilities of adjusting the leg length to match the opposite side through bone growth continue to be offset by the considerable risk of infection due to repeated large surgical access in the area of the prosthesis.

This problem is addressed for the first time by the prosthesis disclosed in EP 3 135 253 A1. The prosthesis described therein has a femoral joint component and a tibial joint component, which are connected to each other in an articulated manner via a joint device and each have through-openings. The through-openings correspond to a guide channel which, de-pending on the knee bending, is aligned with the femoral joint portion or with the tibial joint portion, thus enabling tools, a distraction intramedullary nail or a rod-shaped shaft anchor to be inserted from the outside into both the femur and the tibia without having to disconnect the prosthesis. EP 3 135 253 A1 thus discloses an advantageous idea for the insertion of tools, a distraction intramedullary nail or a rod-shaped shaft anchorage, but the permanent stability of the prosthesis is impaired by the through-openings, as these pass directly through the joint components of the joint device which are in sliding engagement and are intended for force transmission.

The present invention is therefore based on the object of providing a prosthesis for replacing the human knee joint which has the advantages of the prosthesis described in EP 3 135 253 A1 and, in addition, meets the high biomechanical long-term requirements and functional demands placed on a knee joint endoprosthesis.

### SUMMARY

To solve the above-mentioned object and other objects, an implantable prosthesis for re-placement of a human knee joint is provided. The prosthesis comprises a joint device with a joint head and a joint base interacting with the joint head, which are provided for selectively realizing at least one joint movement in the form of a bending and/or extension movement. The joint head of the prosthesis has two first sliding surfaces which are arranged at opposite axial ends of the joint head and are spatially separated from each other by a central joint head section. The joint base has two second sliding surfaces which are formed axially opposite each other on laterally arranged joint elements of the joint base and are in sliding engagement with the respective first sliding surfaces of the joint head, wherein the two first sliding surfaces and the two second sliding surfaces are formed to realize at least one bending and/or extension movement. The central joint head section has at least one guide channel (preferably two guide channels) for passing a tool, a distraction intramedullary nail or a femoral or tibial shaft anchorage.

The joint base is pivotally coupled to the joint head via the axially outwardly displaced first sliding surfaces and second sliding surfaces. Thus, the joint device according to the invention completely decouples the joint components (sliding surfaces) contributing to the pivotal coupling from the central joint head section and the at least one guide channel realized therein. This results in a permanently stable prosthesis that meets the biomechanical long-term requirements and functional demands placed on a knee joint endoprosthesis, while also enabling the advantageous passing of tools, a distraction intramedullary nail, or a rod-shaped shaft anchorage as described in EP 3 135 253 A1.

The prosthesis may further comprise a femoral bone shaft replacement element and/or a tibial bone shaft replacement element for replacing a removed bone. To replace the removed femoral joint, the joint head may be connected to the distal end of the femoral bone shaft replacement element or form a unit therewith. To replace the removed tibial joint, the joint base may be connected to the proximal end of the tibial bone shaft replacement element or form a unit therewith.

The joint head may comprise two outer joint head sections which are spatially separated from each other in the axial direction by the central joint head section; One of the two first sliding surfaces may be formed at the axial outer end of each outer joint head section. This means that the sliding surfaces (joint surfaces) intended for the movable coupling are positioned as far out as possible in the axial direction, thereby achieving a stable articulated coupling even with joint devices of small size/dimensions.

The size/dimensions of the prosthesis depend on the anatomical conditions, particularly in the joint area. In order to achieve a joint device that is as simple and compact as possible, the joint head of the prosthesis can be formed to be essentially roller-shaped (cylindrical) or hollow cylindrical. The cylindrical or hollow cylindrical joint head can have a predetermined axial extension (i.e., extension along its axis of rotation) and a predetermined radial extension (extension perpendicular to its axis of rotation), wherein the extension (dimension) in the axial and radial directions is limited or predetermined by the surrounding soft tissue. Accordingly, the two first sliding surfaces are formed on or near the opposite axial outer end sections of the cylindrical or hollow cylindrical joint head; the central joint head section is formed centrally between the two axial end sections of the cylindrical or hollow cylindrical joint head.

The joint base, which is hinged to the joint head, can be formed to movably receive the (cylindrical or hollow cylindrical) joint head. In particular, the joint base can be formed in a fork shape with two joint elements arranged laterally (medially and laterally) and spaced axially apart from each other. For example, the axially spaced joint elements can be arranged laterally on the outside. They can each be medially or laterally (form-fittingly) connected to the joint base. Further, the lateral joint elements may be arranged further axially outward compared to the outer head sections or axial ends of the joint head; this allows the joint head to be positioned in a space-saving manner in the joint space created between the two laterally arranged joint elements.

Further, the two second sliding surfaces of the joint base can be arranged on the inner sides of the two laterally arranged joint elements so that a second sliding surface can come into sliding engagement with a corresponding first sliding surface at the two opposite axial ends of the joint head. In other words, the joint head and the joint base are rotatably (jointly) coupled to each other via their respective axially outwardly located sliding surfaces. This fork-shaped external mounting of the joint head and joint base increases the stability of the joint device. At the same time, the joint device has no joint components in the center that contribute to the joint coupling. This is because the central joint head section does not contribute to the articulated coupling; it has no joint function, in particular no force-transmitting function. Rather, the central joint head section has a guiding function for the insertion of a tool, a distraction intramedullary nail, and/or a femoral or tibial shaft anchor. In contrast to the joint device described in EP 3 135 253 B1, with the joint device according to the invention, the joint function and the associated force flow are spatially and functionally completely decoupled from the guide function for the anchoring elements.

### Joint Axis-Range of Motion of the Joint Device

Starting from an extended position, the joint device can be formed to realize a bending movement of up to 120°. The bending movement is a rotational movement about a virtual joint axis defined by the movable mounting of the joint head and joint base described above.

Accordingly, “axial” or “axial direction” refers to a direction parallel to the virtual joint axis, and “radial or radial direction” refers to a direction perpendicular to the joint axis.

To limit the rotational movement around the joint axis, in particular the extension movement, the joint device may have an extension stop. The extension stop can be realized by at least one stop surface formed on the joint head toward the ventral side (i.e., toward the front). The at least one stop surface on the joint head can be realized by the cylindrical or hollow cylindrical joint head having a body extension in the radial direction which increases locally (slightly) towards the ventral side of the joint head. In other words, the joint head may not be completely circular on its outer side in the circumferential direction, but may have a local increase in material deviating from the circular course and directed toward the ventral side. This local increase in material (expansion) on the ventral side of the joint head creates a stop surface that can interact with a corresponding stop element on the joint base, thus preventing overextension of the prosthesis.

In order to further increase the effect of the ventral extension stop described here, the virtual joint axis may be shifted (slightly) in the dorsal direction in relation to the center of the femur longitudinal axis.

The joint device can further be formed to allow a limited (restricted) rotational movement around the tibial shaft axis in addition to the bending and/or extension movement described here. In particular, the joint device can be formed to allow the additional limited rotational movement only when the joint device is in a bent position (and thus in the functional position of knee bending), which corresponds to the physiological conditions. In the extended position, however, rotational movement about the tibial shaft axis is blocked by the stop element.

### Formation of the Sliding Surfaces (Joint Surfaces)

The range of motion of the joint device, as described above, can be realized by appropriately forming the two first sliding surfaces (joint surfaces) and the two second sliding surfaces (joint surfaces) corresponding to the two first sliding surfaces.

According to a first embodiment variant, the two first sliding surfaces and the two second sliding surfaces can be sections of a cylindrical surface whose radii (diameters) are matched to each other so that a play-free sliding movement and thus a rotation about the joint axis is possible. In other words, the two first sliding surfaces can each be radially curved sliding surfaces which each have a first radial distance (first radius) from the joint axis (axis of rotation of the joint head) in the circumferential direction. In a sectional view perpendicular to the axis of rotation, the first sliding surfaces can each be circular. Similarly, the two second sliding surfaces can each be radially curved sliding surfaces which, in the circumferential direction, each have a second radial distance (second radius) from the joint axis (axis of rotation of the joint head). In a sectional view perpendicular to the axis of rotation, the second sliding surfaces can each be circular. The second radius of the second sliding surfaces can be matched to the first radius of the first sliding surfaces in such a way that a first sliding surface comes into sliding engagement with a second sliding surface on the medial and lateral sides of the joint device, thus enabling a play-free rotational movement about the joint axis.

For example, the respective first sliding surfaces can be the inner surface of a respective outer ring which is arranged on the opposite outer joint head sections and thus projects axially outwards at the respective axial ends of the joint head. Furthermore, the respective second sliding surfaces can be the outer surface of a respective inner ring which projects axially inwards at the respective opposite lateral joint elements. Alternatively, the respective first sliding surfaces may be the outer surface of a respective inner ring which is arranged on the opposite outer joint head sections and thus projects axially outwards at the respective opposite axial ends of the joint head. Further, the respective second sliding surfaces may be inner surfaces of a respective outer ring which projects axially inwards at the respective opposite lateral joint elements. When the joint device is mounted, the respective inner rings engage with the respective outer rings at both axial ends, causing the first sliding surfaces to come into sliding contact with the second sliding surfaces, respectively. The circularly curved configuration of the first sliding surfaces and second sliding surfaces relative to the joint axis described here allows a rotational movement about the joint axis of the joint device and thus the above-described optional bending and/or extension movement to be achieved.

In order to come as close as possible to the physiological knee joint function, according to a second embodiment variant, the two first sliding surfaces and the two second sliding surfaces can also be radially curved section surfaces of a spherical shell with the virtual sphere center in the center of the knee joint prosthesis, so that, in addition to the bending movement, a defined rotational movement about the longitudinal axis of the leg is also possible.

In other words, in the second embodiment variant, the first sliding surfaces and the second sliding surfaces can each be spherical or at least partially spherical. According to one variant, the two first sliding surfaces can each be (partially) spherically convex and the corresponding second sliding surfaces can each be (partially) spherically concave, thus each forming a partial segment of a spherical surface. For example, the (partially) spherically convex first sliding surfaces can be arranged on the opposite outer joint head sections and thus protrude axially outwards at the respective axial ends of the joint head; the (partially) spherically concave second sliding surfaces, on the other hand, can be recessed on the inner sides of the axially opposite lateral joint elements of the joint base. According to an alternative variant, the two first sliding surfaces can each be (partially) spherically concave and the corresponding second sliding surfaces can each be (partially) spherically convex. In this case, the (partially) spherically concave first sliding surfaces can be recessed at the opposite axial ends of the joint head, while the (partially) spherically convex second sliding surfaces protrude on the inner sides of the axially opposite lateral joint elements of the joint base.

The (partially) spherically convex sliding surfaces may be the surfaces of corresponding projections formed either on the inner sides of the lateral joint elements of the joint base or on the axial ends (axial outer sides) of the joint head. Furthermore, the (partially) spherical concave surfaces may be the surfaces of corresponding grooves formed either on the inner sides of the lateral joint elements of the joint base or on the axial ends (axial outer sides) of the joint head. When the joint device is mounted, the respective (partially) convex spherical sliding surfaces at both axial ends engage in the corresponding, respective (partially) concave spherical sliding surfaces, whereby a (partial) ball bearing arrangement is realized between the joint head and the joint base, which not only allows a rotational movement about the joint axis to realize a bending and extension movement, but also allows a limited rotational movement (rotation of a few degrees) about the longitudinal axis of the leg. This allows the natural mobility of a knee joint to be replicated even better.

In both embodiment variants, the virtual center of rotation of the cylinder or the ball may be shifted slightly in the dorsal direction; this additionally improves the extension stop described above in a beneficial manner. If the sliding surfaces correspond to a spherical shell section, the extension stop can also prevent the joint base from additionally being rotatable perpendicular to the joint axis when in the fully extended position. In other words, the (limited) rotation of the joint base about the tibia axis and thus relative to the joint head about an axis perpendicular to the joint axis is only possible when the joint device is in a bent position (and thus in the functional position of knee bending), which corresponds to the physiological conditions.

Regardless of the specific embodiment variants described here, the two first sliding surfaces and/or the two second sliding surfaces may each have a biocompatible coating to reduce friction. Alternatively, the two first sliding surfaces and/or the two second sliding surfaces may each be made of an additional material component which is wear-resistant and contributes to reducing friction between the joint head and the joint base. Any type of material pairing or surface coating known from the prior art may be used for the first and second sliding surfaces.

### Formation of the Guide Channels

As described above, at least one guide channel is formed in the central joint head section. Preferably, two guide channels are formed in the central joint head section. Each guide channel can be formed as a through-bore in the central joint head section and dimensioned such that a tool, a distraction intramedullary nail and/or a femoral or tibial shaft anchor can be passed through the guide channel. In particular, a guide channel can be arranged (or aligned) in the central joint head section in such a way that it aligns with a through-opening of an adjacent femoral bone shaft replacement element or with a through-opening of the joint base when the joint device achieves a predetermined bending angle. The predetermined bending angle may correspond to an angle which the joint head and the joint base of the joint device assume when a functional position corresponding to knee bending is achieved.

Similar to EP 3 135 253 A1, the predetermined bending angle, at which a guide channel is aligned with a through-opening of an adjacent femoral bone replacement part or the tibial joint part (joint base), can be in the range of 20° to 90°, preferably in the range of 30° to 60°, and particularly preferably in the range of 30° to 45°. The extension position is again taken as a reference for the specified bending angle ranges. The embodiment described here allows a tool, a distraction intramedullary nail or a femoral or tibial shaft anchorage to be inserted in a simple and gentle manner via the respective guide channel into the adjacent through-opening of the femoral and/or tibial joint element or the bone shaft replacement element and further into the respective bone without having to decouple the joint device of the prosthesis. This can significantly reduce the surgical intervention (for example, when replacing a distraction intramedullary nail by a tibial shaft anchor) and significantly lower the risk of infection.

The central joint head section and thus the central part of the knee joint prosthesis there-fore has the sole function of guiding the insertion of a tool, a distraction intramedullary nail and/or a femoral or tibial shaft anchor. In contrast to the joint device described in EP 3 135 253 B1, the joint device according to the invention completely separates the joint function and the guide function for the anchoring elements, both spatially and functionally. The advantageous arrangement of the joint elements as far out as possible also ensures long-term stability.

The femoral joint part (joint head) may further comprise a sliding surface for a kneecap on the ventral side.

### BRIEF DESCRIPTION OF THE DRAWINGS

Further advantages and aspects of the invention are explained with reference to the drawings. These show:

FIG. 1 an external view of a prosthesis according to the invention for replacement of the human knee joint in a slightly bent position;

FIG. 2 an exploded view of the prosthesis shown in FIG. 1;

FIGS. 3a, 3b further views of the prosthesis shown in FIG. 1;

FIG. 4 an exploded view of a further prosthesis according to the invention for replacement of the human knee joint; and

FIGS. 5a, 5b further views of the prosthesis shown in FIG. 4.

### DETAILED DESCRIPTION

Aspects of the present invention are further described below with reference to exemplary embodiments.

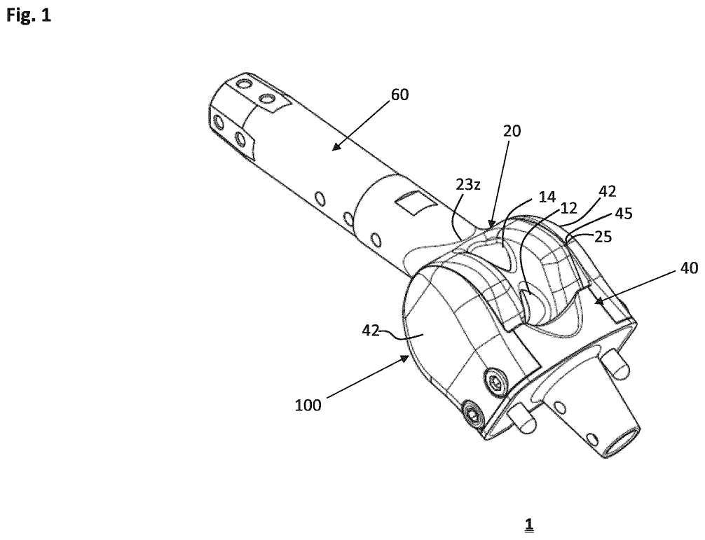

FIG. 1 shows an external view of a prosthesis 1 according to the invention in a slightly bent position.

The prosthesis 1 comprises a joint device 100 with a joint head 20 and a joint base 40 that interacts in a joint manner with the joint head 20. In FIG. 1, the prosthesis 1 is shown with a femoral bone shaft replacement element 60, which is connected at its distal end to the joint head 20 or can also form a unit therewith. Further, the joint base 40 of the prosthesis 1 can be connected to the proximal end of a tibial bone shaft replacement element (not shown) or can also form a unit therewith. Depending on the use of the prosthesis 1, the femoral or tibial bone shaft replacement element can also be omitted.

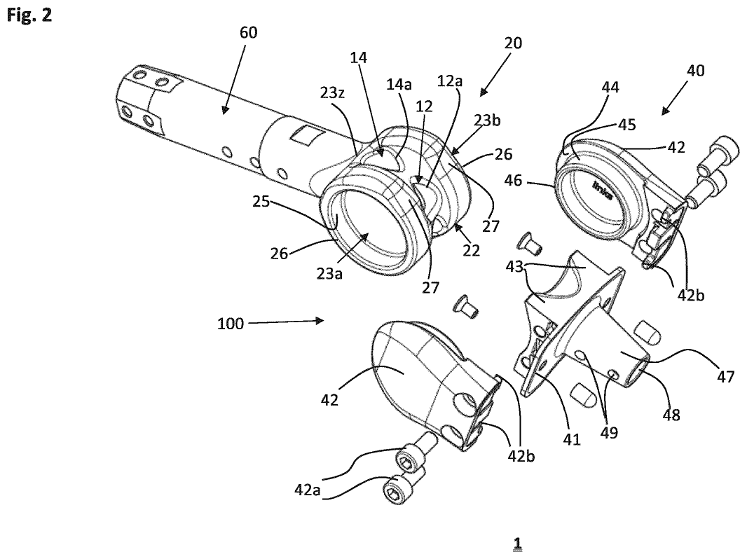

In connection with FIG. 2, the joint device 100 of the prosthesis 1 is described in further detail.

The joint head 20 of the joint device 100 is roller shaped (cylindrical in shape). The cylindrical body 22 of the joint head 20 defines a rotational axis which corresponds to the joint axis. Further, the cylindrical body 22 has a centrally arranged body section (hereinafter referred to as the central joint head section 23z) and two outer body sections (hereinafter referred to as the outer joint head sections 23a/23b), which are spatially separated from each other in the axial direction by the central joint head section 23z. The joint head 20 can be coupled to the femoral bone shaft replacement element 60 via its central joint head section 23z or form a unit therewith or be connected directly to the femur via a shaft anchorage.

The femoral bone shaft replacement element 60 can also have a through-opening (not visible in FIG. 2 because it is concealed). This through-opening can open into a guide channel 12 of the central joint head section 23z.

In the region of the two outer joint head sections 23a, 23b, the joint head 20 has a ring-shaped projection 26, respectively. The two ring-shaped projections 26 each form an outer ring 26 at the opposite axial ends of the joint head (or its cylindrical body 22). The inner surface 25 of the respective outer ring 26 forms a first circular sliding surface 25 (joint surface 25) of the joint device 100.

Further, the joint head 20 comprises at least one stop surface 27 on its radial outer surface. The stop surface 27 is formed on both outer joint head sections 23a, 23b. In terms of design, the stop surface 27 can be realized by the cylindrical or hollow cylindrical joint head 20 having a shape on the outside that deviates locally from a circular or cylindrical body shape. As can be seen from FIGS. 1 and 2, the radial extension of the joint head 20 increases slightly towards the ventral side (i.e. towards the front). This additional radial extension or increase in material of the joint head 20 remains essentially limited to the front side of the joint head 20 in the circumferential direction of the joint head 20, so that the stop surface 27 shown in FIG. 2 is formed there on the two outer joint head sections 23. A slight dorsal displacement of the joint axis of the joint head 20 in relation to the femur shaft axis prevents the unilateral increase in material (or radial expansion) of the joint head 20 from having an anatomically unfavorable effect in the ventral direction and optimizes the joint device 100 in accordance with the requirements.

The joint base 40 of the joint device 100 shown in FIG. 2 comprises two laterally arranged joint elements 42. These lateral joint elements 42 are each arranged at an axial distance from one another on a centrally arranged joint element 41. The axial distance between the two lateral joint elements 42 is selected such that the lateral joint elements 42 are located further out axially relative to the outer joint head sections 23a, 23b. Thus, the joint head 20 is mounted rotatably between the two lateral joint elements 42 about the joint axis, as will be described in more detail below.

The lateral joint elements 42 can be detachably fastened to the central joint element 41 of the joint base 40 by means of screws 42a. Other fastening means are also conceivable. Further, adjusting lugs 42b may be provided on the two lateral joint elements 42, which are formed to engage positively in corresponding adjusting grooves (not visible in FIG. 2) on the central joint element 41. This ensures correct mounting, in particular the correct alignment of the two lateral joint elements 42 with respect to the central joint element 41. Further, the adjusting lugs 42b can be formed as stable form-locking elements to prevent shear forces and bending moments from the screws 42a, thereby further increasing the mechanical stability of the joint device 100 and thus meeting the high demands placed on it. The screws 42a are therefore only subjected to axial forces.

A stop element 43 is also arranged on the central joint element 41 between the two lateral joint elements 42. It is formed to come into contact with the at least one stop surface 27 of the joint head 20 during an extension movement, thereby preventing the joint from being overextended. In other words, the at least one stop surface 27 on the joint head 20 and the stop element 43 define a maximum possible extension position of the joint device 100.

The central joint element 41 can be connected to a tibial bone shaft replacement element (not shown) on its side facing away from the stop element 43 and the two lateral joint elements 42. The joint base 40 has a fixation extension 47 through which a through-opening 48 extends. The through-opening 48 can in turn open into a guide channel 14 formed in the central joint head section 23z. A tibial shaft anchorage (not shown in FIG. 1), a tool or a distraction intramedullary nail, for example, can be passed through the through-opening 48 of the joint base 40 to the tibia. In particular, the distraction intramedullary nail or the tibial shaft anchor can be fastened to the fixation extension 47 by means of fastening pins, by inserting the fastening pins through transverse bores 49 provided in the wall of the fixation extension 47.

The two lateral joint elements 42 together with the central joint element 41 form the fork-shaped joint base 40. The two lateral joint elements 42 each have an axial inner surface 44 on which a ring-shaped projection 46 (hereinafter also referred to as inner ring 46) is formed. The respective annular projection 46 has a circular outer surface 45, which forms the second sliding surface 45 (joint surface 45) of the joint device 100.

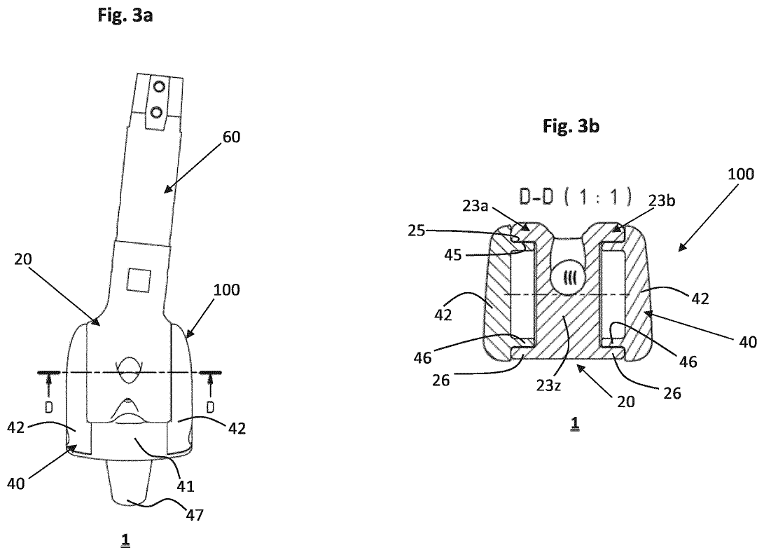

In connection with FIGS. 3a and 3b, the interaction of the respective first and second sliding surfaces (joint surfaces) 25 and 45 of the joint device 100 is described in more detail. FIG. 3a shows a top view of the prosthesis 1 with the joint device 100. FIG. 3b shows a sectional view along the section plane D-D. When mounted, the respective inner rings 46 of the two lateral joint elements 42 engage in the respective outer rings 26 of the joint head 20. As a result, the circularly curved second sliding surfaces 45 of the lateral joint elements 42 come into sliding engagement with the circularly curved first sliding surfaces 25 of the joint head 20. This allows the joint base 40 to be rotated relative to the joint head 20 about the joint axis, thereby enabling a bending or extension movement. To improve the sliding properties, the respective sliding surfaces 25, 45 can each be provided with a biocompatible coating or be made of a wear-resistant and friction-reducing material component known from the prior art.

Back to FIG. 2. The detachable mounting of the two lateral joint elements 42 on the central joint element 41 (this is done using the screws 42a and adjusting lugs 42b described above) allows flexible mounting of the joint device 100 (and thus of the prosthesis 1) during the surgical procedure without compromising stability. This allows the joint device 100 to be mounted from both the medial and lateral sides, depending on the surgical access. In both mounting variants, the prosthesis 1 can be pre-mounted during the surgical procedure, wherein, when inserting the prosthesis 1 from the medial/lateral side, the lateral/medial joint element 42 facing away from the medial/lateral side is first pre-mounted on the central joint element 41. After inserting the prosthesis 1, the joint element 42 facing the medial/lateral side can be attached to the central joint element 41, thereby closing the joint device 100.

The embodiment of the joint device 100 described herein, in which the articulated bearing between the joint head 20 on the one hand and the fork-shaped joint base 40 on the other hand is displaced axially as far outwards as anatomically possible, improves the overall stability of the joint device 100. The central joint head section 23z, in which the guide channels 12, 14 are provided for the passage of a tool, distraction intramedullary nail or a femoral or tibial shaft anchor, has no joint-stabilizing function.

The joint device 100 is generally formed to perform a bending and/or extension movement corresponding to a natural knee joint. This is achieved by rotation/turning movement of the joint base 40 relative to the joint head 20 about the joint axis. Preferably, the joint device 100 can achieve a maximum bending angle of 120°starting from an extension position. The extension position is then reached during the rotational movement when the central joint element 41 of the joint base 40 comes into contact with at least one stop surface 27 of the joint head 20.

The ventral side of the joint head 20 can further be configured to form a sliding surface for the kneecap. The outer openings 12a and 14a of the guide channels 12 and 14 are located in an area of the joint head 20 that does not come into contact with the lower surface of the kneecap, so that the movement of the kneecap is not impaired.

The guide channels 12 and 14 (see FIG. 2) are realized in the form of through-bores in the central joint head section 23z with outer openings 12a, 14a formed toward the ventral side. The guide channels 12 and 14 (through-bores) are arranged in the central joint head section 23z in such a way that each guide channel 12, 14 communicates with a through-opening of the femoral bone shaft replacement element 60 or with the through-opening 48 of the joint base 40 and, if applicable, the tibial bone shaft replacement element (not shown in FIG. 2) when the prosthesis 1 or its joint device 100 realizes a predetermined bending position (pre-determined bending angle) corresponding to knee bending.

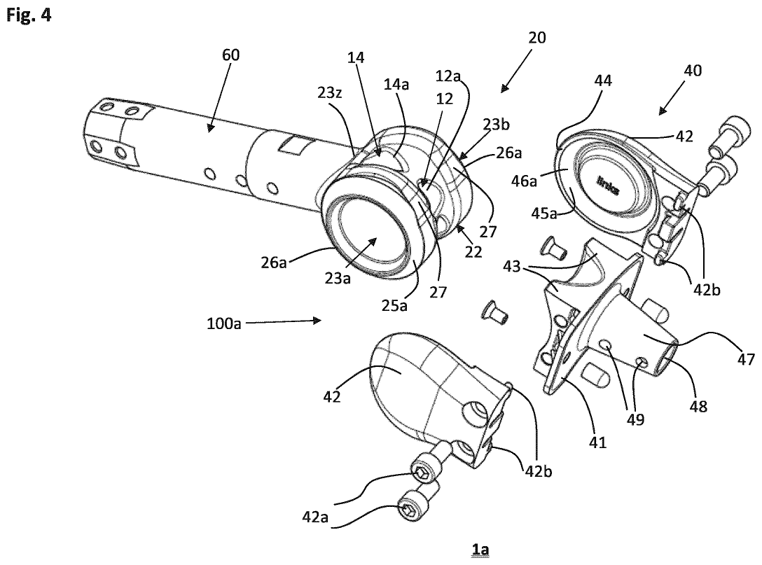

In connection with FIG. 4, a further variant of a prosthesis 1a according to the invention with a joint device 100a for replacing the human knee joint is described.

FIG. 4 shows an exploded view of the prosthesis 1a. It differs from the prosthesis 1 according to FIGS. 1, 2, 3a, and 3b in the formation of the first and second sliding surfaces of the joint device 100a and thus in the articulated coupling between the joint head 20 and the joint base 40. All other components of prosthesis 1a and joint device 100a are structurally and functionally identical to prosthesis 1 and joint device 100 in FIGS. 1, 2, 3a, and 3b, so that reference is made to the above description to avoid unnecessary repetition. The following description only describes the differences from the joint device 100 of the prosthesis 1 according to FIGS. 1, 2, 3a, and 3b.

In contrast to the embodiment shown in FIGS. 1, 2, 3a and 3b, the joint head 20 comprises a ring-shaped projection 26a at each of its opposite axial ends, with a convexly curved, spherical surface 25a corresponding to a section (segment) of a spherical surface. The convexly curved, spherical surface 25a acts as the first sliding surface 25a of the joint head 20.

Further, the two axially spaced lateral joint elements 42 of the fork-shaped joint base 40 each have a ring-shaped groove 46a on their axial inner surfaces 44 corresponding to the ring-shaped projections 26a. The respective annular groove 46a has a concave, spherical surface 45a, which acts as a second sliding surface 45a and corresponds to a section (segment) of a spherical surface.

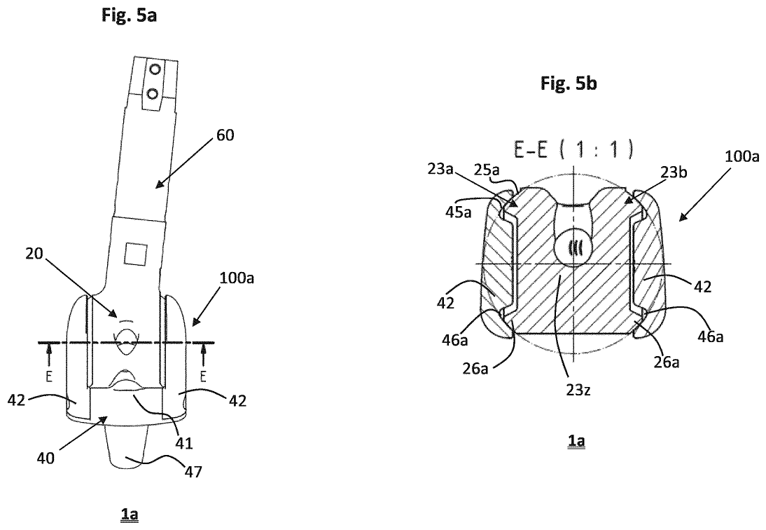

In connection with FIGS. 5a and 5b, the interaction of the respective first and second sliding surfaces (joint surfaces) 25a and 45a of the joint device 100a is described in more detail. FIG. 5a shows a top view of the prosthesis 1a with the joint device 100a. FIG. 5b shows a sectional view along the section plane E-E. When mounted, the annular projections 26a arranged at the axial ends of the joint head 20 engage in the annular grooves 46a arranged on the axial inner surfaces 44 of the lateral joint elements 42 and opposite the annular projections 26a. This causes the spherically convex first sliding surfaces 25a of the joint head 20 to come into sliding contact with the respective spherically concave second sliding surfaces 45a of the joint base 40 (see FIG. 5b).

This embodiment not only allows rotation about the joint axis, but also limited rotation (by a few degrees) about a rotation axis arranged perpendicular to the joint axis and passing through the joint base 40. This additional rotation is particularly possible when the joint device 100a is in a bent position. If, on the other hand, the joint device 100a is in the fully ex-tended position, in which the stop element 43 of the joint base 40 is in contact with the at least one stop surface 27 of the joint head 20, this additional rotation is prevented by the interaction of the stop element 43 with the at least one stop surface 27. The additional slight rotation perpendicular to the joint axis in the bent position and the elimination of rotation in the extended position described here allow the mobility of a knee joint to be reproduced even more physiologically, as a limited rotational movement around the tibia axis is possible when the knee is bent.

### LIST OF REFERENCE SIGNS

1,1a prosthesis
12 first through-hole/first guide channel
12a outer opening
14 second through-hole/second guide channel
14a outer opening
20 joint head
22 cylindrical body
23a, 23b outer joint head sections
23z central joint head section
25, 25a first sliding surface(s)
26 outer ring
26a ring-shaped projection
27 stop surface(s)
40 joint base
41 central joint element
42 side joint element(s)
42a screws
42b adjusting lugs
43 stop element
44 axial inner surface(s) of the side joint element(s)
45,45a second sliding surface(s)
46 inner ring
46a ring-shaped groove
47 fixing extension
48 through-opening joint base
49 transverse bore
60 femoral bone shaft replacement element
100, 100a joint device

1. An implantable prosthesis for replacement of a human knee joint, comprising:
a joint device with a joint head and a joint base interacting with the joint head, (which are provided for selectively realizing at least one joint movement in the form of a bending and/or extension movement,
wherein the joint head has two first sliding surfaces which are arranged at opposite axial ends of the joint head and are spatially separated from each other by a central joint head section
wherein the joint base has two second sliding surfaces which are formed axially opposite each other on laterally arranged joint elements of the joint base and are in sliding engagement with the respective first sliding surfaces of the joint head, wherein the two first sliding surfaces and the two second sliding surfaces are formed to realize at least one bending and/or extension movement, and
wherein the central joint head section has at least one guide channel for passing a tool, a distraction intramedullary nail or a femoral or tibial shaft anchor.

2. The implantable prosthesis, according to claim 1, wherein the prosthesis comprises a femoral bone shaft replacement element and/or a tibial bone shaft replacement element, wherein the joint head is connected to the distal end of the femoral bone shaft replacement element or forms a unit therewith, wherein the joint base is connected to the proximal end of the tibial bone shaft replacement element or forms a unit therewith.

3. The implantable prosthesis, according to claim 1, wherein the joint head comprises two outer joint head sections which are spatially separated from each other in the axial direction by the central joint head section wherein one of the two first sliding surfaces is formed at the axial outer end of each outer head section.

4. The implantable prosthesis according to claim 1, wherein the joint head is essentially cylindrical in shape.

5. The implantable prosthesis, according to claim 1, wherein the joint base is formed to movably receive the joint head.

6. The implantable prosthesis according to claim 5, wherein the joint base is fork-shaped with two joint elements formed to movably receive the joint head, which are arranged on the joint base at an axial distance from each other.

7. The implantable prosthesis according to claim 6, wherein the two second sliding surfaces are formed on the inner sides of the two laterally arranged joint elements.

8. The implantable prosthesis according to claim 1, wherein the joint device is formed to realize a bending movement of up to 120° starting from an extended position.

9. The implantable prosthesis, according to claim 1, wherein the joint device further comprises an extension stop, which is provided to limit the extension movement, wherein the extension stop is realized by at least one stop surface formed on the joint head.

10. The implantable prosthesis according to claim 1, wherein the two first sliding surfaces and the two second sliding surfaces are formed to enable, in addition to the bending and/or extension movement about the joint axis, a limited rotational movement perpendicular thereto when the joint device is in a bent position.

11. The implantable prosthesis according to claim 1, wherein the two first sliding surfaces and the two second sliding surfaces are each spherically curved sliding surfaces.

12. The implantable prosthesis, according to claim 1, wherein the two first sliding surfaces and the two second sliding surfaces are each circularly curved sliding surfaces.

13. The implantable prosthesis according to claim 1, wherein the two first sliding surfaces and/or the two second sliding surfaces have a biocompatible coating or consist of an additional material component for reducing friction.

14. The implantable prosthesis, according to claim 1, wherein the joint device further comprises a sliding surface for a kneecap.

15. The implantable prosthesis according to claim 1, wherein the at least one guide channel is arranged in the central joint head section in such a way that a tool, a distraction intramedullary nail or a femoral or tibial shaft anchor can be passed through the joint device at a predetermined bending angle into an adjacent femoral joint head, an adjacent femoral bone shaft replacement element and/or a tibial joint base, a tibial bone shaft replacement element.
